(19) United States
(12) Patent Application Publication
(10) Pub. No.: US 2007/0142814 A1
(43) Pub. Date: Jun. 21, 2007

(54) Foldable garment storage

(76) Inventors: Victoria Akselrod, Rehovot (IL); Rami Gabay, Qiryat-Gat (IL); Moshe Gadot, Rehovot (IL)

Correspondence Address: PEARL COHEN ZEDEK LATZER, LLP, 1500 BROADWAY 12TH FLOOR, NEW YORK, NY, 10036, US

(21) Appl. No.: 11/638,498

(22) Filed: Dec. 14, 2006

Related U.S. Application Data
(60) Provisional application No. 60/750,399, filed on Dec. 15, 2005.

(51) Int. Cl. A61F 13/15

(52) U.S. Cl. 604/387

(57) ABSTRACT

A system and method of holding a garment on a hanger having suspended panels, such that when the panels are folded, a hung garment is also held in a folded state.

### CROSS-REFERENCE FROM RELATED APPLICATION

This application claims the benefit of U.S. Provisional Patent Application 60/750,399 filed on Dec. 15, 2005 and entitled Foldable Garment Storage, a copy of which is incorporated herein by reference in its entirety.

### FIELD OF THE INVENTION

The present invention relates generally to storage of garments such as suits, shirts or jackets.

### BACKGROUND OF THE INVENTION

Packing a garment such as a jacket or suit for travel typically entails either putting the suit into a suit bag that may be separate from a valise or suitcase, or folding the suit or jacket into a suitcase that may include a plethora of other items. Traveling with a separate suit or garment bag can be inconvenient and burdensome. Packing a suit into a crowded valise or suitcase frequently results in unwanted creases to the suit such that the suit arrives in need of pressing or ironing.

### SUMMARY OF INVENTION

Some embodiments of the invention may include a garment holder having a hanger to hold a jacket; and panels having a front face and a back face, where a lower panel is moveably attached to a higher panel so that a front face of the lower panel is foldable to face a front face of the higher panel; so that when a jacket hangs on the hanger, the jacket is folded when the lower panel is folded to face the higher panel.

In some embodiments, the holder may include a handle for carrying the holder and a hanging hook for hanging the holder.

In some embodiments, the holder may include an indentation into which the hanging hook may be inserted when the hanging hook is lowered below a top of the garment holder.

In some embodiments, a handle may be attached to the holder by a member to raise the handle above a top of the hanger and lower the handle below the top of the hanger.

In some embodiments, the hanging hook may be attached to the holder by way of a rotatable member to raise the hanging hook above a top of the hanger and lower the garment hook below the top of the hanger.

In some embodiments the holder may include a case suitable to cover the holder when the panels are folded. In some embodiments the case may include a lip to pass through an aperture of the handle to secure a first side of the case to a second side of the case. In some embodiments, the handle may protrude out of the case when the case covers the garment holder. In some embodiments, a panel may include a pouch or container for holding the case.

In some embodiments a panel may be connected to another panel by a pliable suspenders to suspend a panel to another panel or to suspend a panel to the hanger.

In some embodiments, the holder may include a rod to hold pants, so that the pants are folded when the lower panel is folded to face the higher panel.

Some embodiments may include a garment holder having a hanger to hold an upper portion of a jacket and foldable panels suspended from the hanger to support a horizontal shape of a lower portion of the jacket, so that when the panels are folded, a lower portion of the jacket is folded to an upper portion of the jacket. In some embodiments, the holder may include a hanging hook and a handle, where the hanging hook can be lowered into a stored position.

Some embodiments may include a method of hanging an upper portion of a jacket on a hanger, and suspending a panel from the hanger, where the jacket is wrapped around the panel; and folding the lower portion of the jacket wrapped around a panel upwards towards the upper portion of the jacket.

### BRIEF DESCRIPTION OF THE DRAWINGS

Embodiments of the invention are illustrated by way of example and not limitation in the figures of the accompanying drawings, in which like reference numerals indicate corresponding, analogous or similar elements, and in which:

FIG. 1A is a conceptual illustration of a garment holder, in accordance with some embodiments of the invention;

FIG. 1B is a conceptual illustration of a hanger hook and handle of a garment holder in accordance with an embodiment of the invention;

FIGS. 2A and 2B are conceptual illustrations of a case to store a garment in accordance with an embodiment of the invention;

FIG. 3 is a conceptual illustration of a bendable garment bolder in accordance with some embodiments of the invention;

FIG. 4 is a conceptual illustration of a panel of a garment holder in accordance with an embodiment of the invention; and

FIG. 5 is a flow diagram of a method in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION OF THE INVENTION

In the following detailed description, numerous specific details are set forth in order to provide a thorough understanding of embodiments of the invention. However it will be understood by those of ordinary skill in the art that the embodiments of the invention may be practiced without these specific details. In other instances, well-known methods, procedures, and components have not been described in detail so as not to obscure the embodiments of the invention.

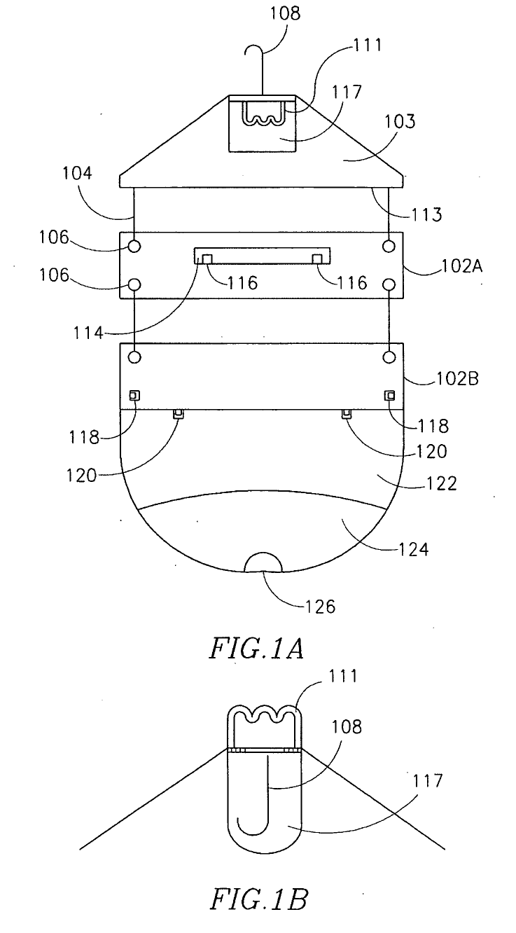

Reference is made to FIG. 1A, a conceptual illustration of a foldable garment holder, in accordance with some embodiments of the invention. In some embodiments, foldable garment holder 100 may include several panels 102, such as for example two panels 102A-102B. Other number of panels 102, such as three or more may be used. Panels 102 may be constructed from a flexible, rigid or semi-rigid material such as for example a bendable plastic or plastic coated cardboard and may support or hold a horizontal shape of a jacket. In some embodiments, panels 102 may be constructed of for example polypropylene with dimensions of 43 cm, measured horizontally, by approximately 25 cm, vertically, such that the width of the panels 102 should be approximately equal to the width of the jacket when folded around the hanger. In some embodiments, a first panel 102A may be larger and/or higher than a second or other panel. Other dimensions and sizes are possible. In some embodiments, the number and dimensions of panels 162 may reflect the size of a jacket or suit to be stored, such that a large suit may be stored in a holder 100 with for example three panels 102 having large dimensions, while a smaller suit may be stored in a holder 100 with two panels 102 having smaller dimensions. Other combinations are possible.

In some embodiments, holder 100 may include a hanger 103 to hold an upper portion of a jacket. Hanger 103 may include pants rod 113, and panels 102 may be attached to or suspended from pants rod 113. In some embodiments, a first panel 102A may be part of or included in the same unit as hanger 103. In some embodiments, panels 102 may be detachable from hanger 103, and may be interchanged so as to be attached to a different hanger 103.

In some embodiments, hanger 103 may include a hanger hook 108 and a handle 111. Hanger hook 108 and handle 111 may be moveably attached to for example one or more hinges or rotating member of hanger 103 so that one or both of hanger hook 108 and handle 111 may be raised or extended above a top of hanger 103 and lowered below a top of hanger 103. In this way, and referring to FIG. 1B, in some embodiments, hanger hook 108 may be lowered or flipped into an indentation 117 in hanger 103 so that hanger hook 108 is stored out of sight or out of the way, and holder 100 may be carried by handle 111 when handle 111 is raised above a top of hanger 103. Some embodiments may not include an indentation 117 or a handle 111.

In some embodiments, one or both of hanger hook 108 and handle 111 may be locked into place, for example in an extended position above the top of hanger 103, by for example clicking an indentation at for example a base of the hanging hook 108 or handle 111 into a flange or protrusion at the top of hanger 103. In some embodiments both hanging hook 108 and handle 111 may assume a raised or lowered position concurrently and independent of each other.

In some embodiments, one or more panels 102 may be connected by for example one or more chains, straps 104, ribbons, links, strings or other flexible or partially flexible or pliable suspenders that may be for example threaded through loops or holes 106 in panels 102. In some embodiments there may be fewer or no straps 104. Other materials may be used for straps 104, and other ways of attaching straps 104 to panels 102 are possible. A strap 104 may connect one panel 102 to another so that the panels 102 may hang vertically when the holder 100 is extended or hung, and so that the holder 100 may be folded between the panels 102 when the holder 100 is to be packed or stored.

In some embodiments, a lower panel 102B may be folded upwards so that a front face of panel 102B faces a front face of panel 102A. Such lower panel 102B may then be folded to face a higher panel 102A. A back face of panel 102B may be folded upward to meet a front face of panel 102A. Other folding combinations are possible and a front or back face of a first panel 102 may be folded to meet a front or back face of another panel 102. In some embodiments one or more of a first panel 102A and second panel 102B may be folded up to the level of hanger 103, so that the panels 102 face some or all of the body of hanger 103.

In some embodiments, one or more panels 102 may include a slit 114 or gap into which one or more pants may be inserted and held in holder 100, such as in panel 102A. In some embodiments, one or more tabs or clasps 116 may be included in or attached to a panel 102A to bold pants securely in slit 114.

In some embodiments, one or more clasps or tabs 118 may be added to or constructed in a panel 102A or 102B or 102C or elsewhere to for example hold one or more sleeves of a jacket in place so that sleeves are not subject to more folding or creases than is necessary. In some embodiments a tab or clasp 120 may be added to a lower panel 102B to hold for example a bottom of a jacket in place on holder 100. Other tabs or clasps 120 may be added in other areas.

In some embodiments, a holder 100 may include a cover 122 that may be attached to for example an upper panel 102A, a lower panel 102B or to some other point of the holder 100. The cover may be slipped over or around the holder 100 when for example the holder 100 is closed or folded. In some embodiments cover 122 may include a flap or pouch 124 that may extend over the top or bottom of the holder 100 so that both sides of the holder 100 may be fully or partially covered by cover 122 when panels 102 are folded one over the other. In some embodiments, cover 122 may be constructed fully or partially of a waterproof material or of a mesh fabric that may be partially stretchable and light-weight, but through which a suit or jacket may be seen from outside of holder 100. In some embodiments, cover 122 or pouch 124 may include a slit 126 through which hanger 103 may be inserted so that for example holder 100 may be hung while holder is covered by cover 122.

In some embodiments, holder 100 may be constructed of cardboard Or other inexpensive and readily disposable materials. In some embodiments holder 100 may be included or sold with a suit or garment.

In some embodiments, an upper portion of a garment may be hung over a top of hanger 103 and a pair of pants may be slipped through slit 114 or over rod 113. Panels 102A and 102B may be folded, for example accordion style over each other and/or up to a level of hanger 103, so that for example the folded holder 100 is approximately as high as a single panel or as hanger 103. In some embodiments, cover 122 may be folded up from a bottom panel 102B to cover the folded panels 102. Pouch 124 may be extended over the top of folded holder 100 and secured over hanger 103 or to a back of holder 100. Other methods of closing or folding holder 100 are possible.

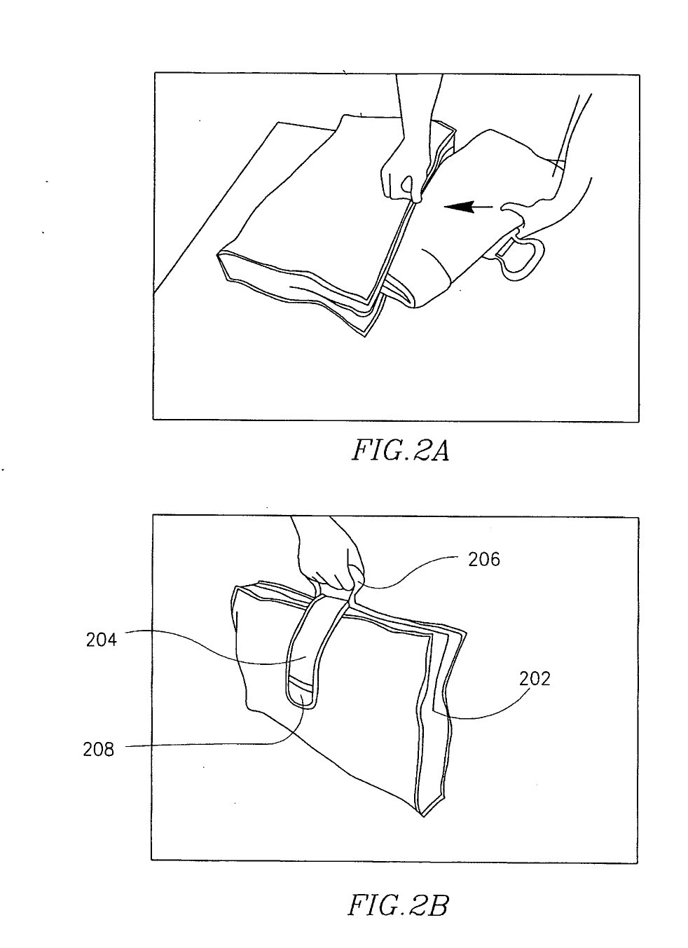

Reference is made to FIGS. 2A and 2B, conceptual illustrations of a case to store a garment holder in accordance with an embodiment of the invention. In FIG. 2A a case 200 may contain or enclose a garment that is folded around a holder 100. Case 200 in FIG. 2B may include a lip 204 or extension of one side of case 200 that may be for example threaded through handle 206, from a first side of case 200 to a second side of case 200, such that lip 204 may hold case 200 in a closed position. Lip 204 may include a clasp 208 such as a buckle, VELCRO™ or other fastener by which lip 204 may be attached to a second side of case 200. In some embodiments, case 200 may be closed without either of handle or hanging hook extending outside of case 200.

Case 200 may be manufactured of any suitable material such as for example, vinyl, leather, cloth, plastic or otherwise. In some embodiments, case 200 may be closed by folding the material in a prescribed way, and tucking tabs of case 200 into corresponding slits of case 200. In some embodiments, case 200 may be closed by a zipper 202, button, or other suitable fashion.

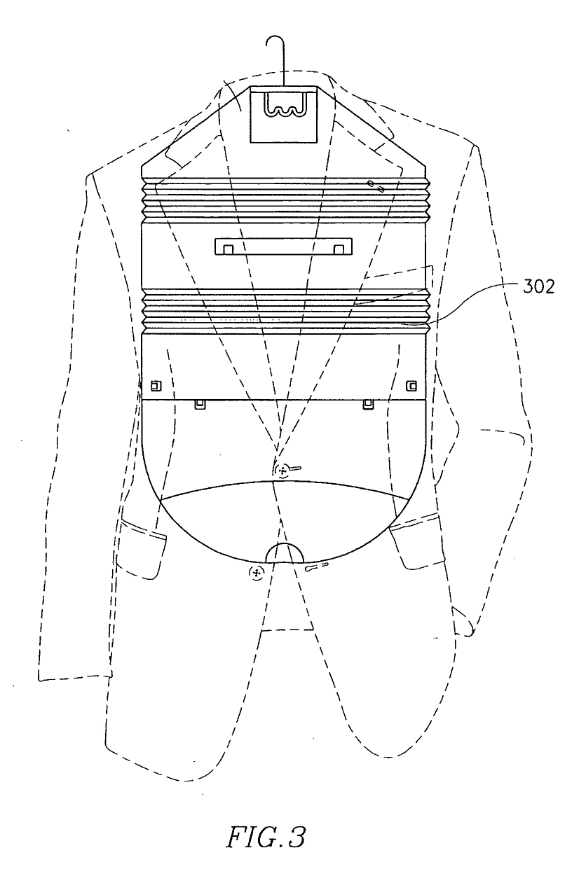

Reference is made to FIG. 3, a conceptual illustration of a bendable garment holder 300 in accordance with some embodiments of the invention. In some embodiments panel 302 may be constructed of one or more panels 302 that may bendable rather than foldable for example along two or more lateral folding areas 304. A bendable panel 302 may in some embodiments minimize creases to a garment 306 or suit that may be caused by folding. Folding areas 304 may be or include for example an area of for example ribbed plastic that may for example bend into a partially folded position without creating a defined fold in a garment. The absence of a defined fold may prevent an unwanted crease from being made in the suit or pants being stored in the holder 300.

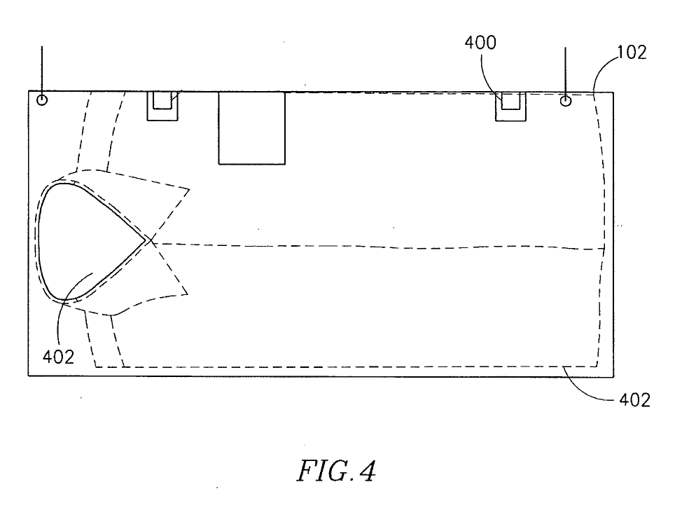

Reference is made to FIG. 4, a conceptual illustration of a panel of a garment holder that may be configured to store one or more shirts in accordance with an embodiment of the invention. In some embodiments, a shirt, such as for example a dress shirt, may be attached to a panel 102. Other garments may be stored. Panel 102 may include tabs 400 or clasps into which may be inserted a part of a shirt such as for example a shoulder corner or bottom edge of a shirt. In some embodiments, a front and back panel of a shirt may be attached to a first side of panel 102, such that the edges of the shirt are held by tabs 400. The sides of a shirt may be folded around to the back of panel 102 and may be held by for example tabs 400 on a back side of panel 102. In some embodiments, sleeves of a shirt may likewise be folded around either a first or second side of panel 102. In some embodiments, panel 102 may include a protrusion 402 around or over which may be attached a collar of a shirt fastened to panel 102. In some embodiments, one or more shirts may be attached to a first side of panel 102 and one or more shirts may be attached to a second side of panel 102.

In some embodiments, one or more panels may include or have attached a pouch, bag 404 or other holder that may be used to store for example a shirt, socks, tie or other accessories. In some embodiments, bag 404 may be used to store case 200 when case is not in use. Bag 404 may be constructed of for example a mesh, cloth or other suitable flexible and lightweight material.

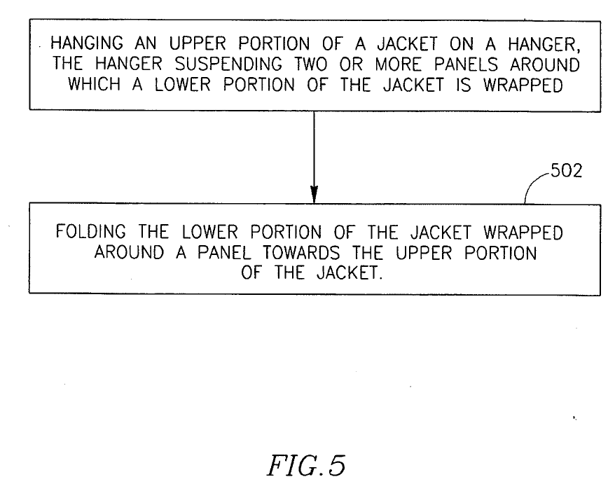

Reference is made to FIG. 5, a flow diagram of a method in accordance with an embodiment of the invention. In block 500, a jacket may be hung over a hanger such that the upper portion of the jacket is wrapped around the hanger, and a lower portion of the jacket wraps around panels that are suspended from the hanger such as from a pants rod of the hanger. In block 502, one or more of the panels may be folded up towards the hanger so that the lower portion of the jacket that is wrapped or extended around the panels is also folded upwards towards the top of the jacket. In some embodiments, pants can be hung over for example a panel or a pants rod, and the pants can also be folded when the panels are folded.

In some embodiments, a hanging hook may be extended above the hanger; and a carrying handle may be flipped below the top of the hanger. In some embodiments, the hanging hook may be flipped into an indentation in the hanger so that in a lowered state it is stored into the hanger body.

In some embodiments the holder may include an attached or separate case that may envelope said hanger and the folded panels. The case may include a lip that extends from one upper end of the case, and that can be threaded through the handle and fastened to another side of the case to secure the case in a closed position.

In some embodiments, panels may be attached to and detached from the hanger so that panels may be interchanged between one hanger and another.

In some embodiments, one or more sleeves of the jacket may be folded in front of the jacket and secured by for example a tab or other fastener to a panel, so that the sleeve stays in place when the panels are folded.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the spirit of the invention.

1. A garment holder comprising:
a plurality of panels suitable to be attached to a hanger, said plurality of panels having a front face and a back face, a second of said plurality of panels moveably attached to a first of said plurality of panels so that a front face of said second panel is foldable to face a front face of said first panel;
wherein when a jacket hangs on said hanger, said jacket is folded when said second panel is folded to face said first panel.

2. The garment holder as in claim 1, comprising said hanger, said hanger having a handle for carrying said holder and a hanging hook for hanging said hanger.

3. The garment holder as in claim 2, comprising an indentation in said hanger into which said hanging hook may be inserted when said hanging hook is lowered below a top is of said garment holder.

4. The garment holder as in claim 2, wherein said handle is attached to said garment holder by a member to raise said handle above a top of said hanger and lower said handle below said top of said hanger.

5. The garment holder as in claim 2, wherein said hanging hook is attached to said garment holder with a rotatable member to raise said hanging hook above a top of said hanger and lower said garment hook below said top of said hanger.

6. The garment holder as in claim 1, comprising a case suitable to cover said garment holder when said panels are folded.

7. The garment holder as in claim 6, comprising a handle on said hanger, wherein said case comprises a lip to pass through an aperture of said handle to secure a first side of said case to a second side of said case.

8. The garment holder as in claim 6, comprising a handle on said hanger that protrudes out of said case when said case covers said garment holder.

9. The garment holder as in claim 1, comprising a plurality of pliable suspenders to suspend said first panel from said hanger.

10. The garment holder as in claim 1, comprising a hanger, said hanger having a rod to hold pants, wherein said pants are folded when said second panel is folded to face said first panel.

11. The garment holder as in claim 1, wherein a panel of said plurality of panels comprises a pouch suitable for holding a case, said case to hold said garment holder when said garment holder is folded.

12. A garment holder comprising:
a plurality of foldable panels suspended for attaching to a hanger, said panels to support a horizontal shape of a lower portion of a jacket, wherein when said panels are folded, said lower portion of said jacket is folded to said upper portion of said jacket.

13. The garment holder as in claim 12, comprising said hanger, said hanger having a hanging book and a handle, wherein said hanging hook is lowerable into a stored position, and said handle is extendable into a raised position.

14. The garment holder as in claim 13, comprising a cover to enclose said hanger and said folded panels.

15. A method comprising:
hanging an upper portion of a jacket on a hanger, said hanger suspending a plurality of panels around which a lower portion of said jacket is wrapped; and
folding said lower portion of said jacket wrapped around a panel of said plurality of panels upwards towards said upper portion of said jacket.

16. The method as in claim 15, comprising extending a hanging hook above said hanger; and lowering a carrying handle below a top of said hanger.

17. The method as in claim 16, comprising fitting said hanging hook into an indentation in said hanger.

18. The method as in claim 15, comprising:
enveloping said hanger and said folded panels in a case.

19. The method as in claim 15, comprising hanging pants over said panels; and folding said pants as said panels are folded.

20. The method as in claim 15, comprising detaching said panels from said hanger. said jacket and attaching said sleeve to a tab in a panel of said plurality of panels.
